(19) United States
(12) Patent Application Publication
(10) Pub. No.: US 2025/0059688 A1
(43) Pub. Date: Feb. 20, 2025

(54) A Mycelium Process Tank System

(71) Applicant: ECCO Sko A/S, Bredebro (DK)

(72) Inventors: Teo Pazanin, Bredebro (DK); Makis Sachperoglou, Bredebro (DK); Max Gilet, Bredebro (DK)

(21) Appl. No.: 18/723,282

(22) PCT Filed: Dec. 16, 2022
(86) PCT No.: PCT/DK2022/050285

§ 371 Date: Jun. 21, 2024

(30) Foreign Application Priority Data
Dec. 23, 2021 (DK) PA 2021 70666

(51) Int. Cl. D04H 1/4266; D06B 5/22; D06B 23/14

(52) U.S. Cl. CPC D04H 1/4266; D06B 5/22; D06B 23/14

(57) ABSTRACT

The invention relates to a mycelium process tank system, the process tank system comprising:
at least one mycelium support base,
a mycelium storing volume in a process tank,
a liquid storing volume fluidly coupled to and/or being a part of the process tank,
the liquid storing volume designating a volume exterior to physical boundaries of the mycelium storing volume,
the mycelium process tank comprising one or more pumps controlled by a pump controller, the one or more pumps being arranged for circulation of process liquid outside the mycelium storing volume in the process tank.

### FIELD OF THE INVENTION

The present invention relates to a method of processing mycelium into a non-woven fabric.

### BACKGROUND OF THE INVENTION

It is well-known that mycelium may be applied for different kinds of purposes. Different processes may be applied depending on the intended mycelium properties and applications.

A challenge within the field of manufacturing of objects and fabrics, e.g. non-woven fabrics, are strength properties. This has been addressed within the art e.g. by including scaffolds or other types of reinforcement with mycelium fabric. Such scaffolding may e.g. included silicone based meshed giving both strength and flexibility to the resulting composite. Such solutions have many benefits, but a challenge with such approaches is that scaffolds may delaminate from the mycelium and also that the scaffold material are typically made from materials which are less biodegradable than desired.

A further way of addressing this issue, including addressing a proper industrially applicable manufacturing with predictable outcome, is to focus on the manufacturing process of the mycelium raw material applied. Growth medium, mixtures of different types of such, controlling of the growth process conditions, such as temperature, humidity etc.

U.S. Pat. No. 10,687,482 B2 illustrates one of such methods and discloses a controlled growth of fungal tissue through a porous material to form a material which may easily be delaminated and subsequently applied for its intended purpose.

A challenge with such approaches, including the above mentioned, is that such methods may not necessarily result in the desired properties and that the resulting produced fabric is either too expensive to make and/or the resulting strength and flexibility is less than desired if the fabric is to be used within applications such as clothing and footwear, where stress conditions are significant.

Within the particular applications mentioned above, it should be noted that the expectations of users of footwear and other wearables—and that such types of items is resistant to stress, possesses a degree of flexibility and are nice to look at. Also after a period of use.

### SUMMARY OF THE INVENTION

The invention relates to a mycelium process tank system, the process tank system comprising at least one mycelium support base, a mycelium storing volume in the process tank, a liquid storing volume fluidly coupled to and/or being a part of the process tank, the liquid storing volume designating a volume exterior to physical boundaries the mycelium storing volume, the mycelium process tank comprising one or more pumps controlled by a pump controller, the one or pump being arranged for circulation of process liquid outside the mycelium storing volume in the process tank.

In the present context establishing of a liquid flow in the exterior process liquid/the process liquid outside the mycelium storing volume facilitates a process where as little as possible mechanical stress is induced to the mycelium material during the mycelium process, whether it is a mycelium wetting process, a mycelium plasticizing process, a mycelium tanning process, a mycelium dyeing process, etc.

The inventive mycelium process tank involves the use of gentle measures to perform the intended process, all involving that the mycelium processed should be either liquid saturated or at least being in the process of being saturated by liquid.

The process liquid may include water or water mixed with relevant process agents.

This means in practice that uncontrolled mechanical stress may as penetration of the mycelium with liquid may be performed without significant stress. Such stress may result in damaging of the mycelium.

As for the process liquid and the definitions regarding to location of this process liquid, it is noted that the location of process liquid during the active induction of flow in the exterior liquid is changing all the time, both due to the active establishment of liquid flow in the exterior process liquid but also due to passive movement of process liquid.

The exact location and the amount process liquid is therefore intentionally changing during wetting, de-wetting, rinsing and/or chemical treatment of the mycelium unless a desired equilibrium has been obtained.

The inventive process is e.g. beneficial for the purpose of obtaining a gently way of transporting process liquid into the mycelium material to obtain the desired penetration of process liquid.

The inventive process benefits in that it is possible to avoid a tumbling process which is typically applied in a tanning process. A tumbling process represents a type of mechanical stress which in uncontrolled and the process may very likely damage the mycelium fibres. It is thus preferred that any mechanical process applied for assisting the present way of transporting process liquid into/through the mycelium fibres is carefully and predictably controlled so as to ensure that optional damage to the mycelium fibres are avoided or kept at a low level.

In an embodiment of the invention the liquid storing volume is at least 20% larger than the mycelium storing volume,

According to an embodiment of the invention, the inducement of active flow over a duration of time, will promote penetration of liquid into the mycelium and thereby promote an efficient plasticising, whether or not the active flow of process liquid is primarily water during an initial wetting process prior to plasticising of mycelium material.

The active flow established is in particular facilitating a uniform penetration of process liquid into the mycelium material and thereby promoting a uniform strength throughout the cross-section of a final fabric. This is in particular relevant in the field of fabrics based on mycelium material, as the starting material is brittle and susceptible to breaking of not properly plasticized.

According to an embodiment of the invention, the process liquid located outside the mycelium storing volume is referred to as exterior process liquid and where process liquid contained inside the mycelium storing volume is referred to as interior process liquid.

According to an embodiment of the invention, a part of the process liquid forming interior process liquid being located within physical boundaries of the mycelium material positioned in the mycelium storing volume.

The part of interior process liquid has a certain value from the beginning of the wetting process and the interior process liquid will increase during the wetting process, at least partly helped by the liquid flow established in the exterior process liquid. The flow patterns, planar waves, circular or tubular flow patterns in the exterior process liquid may thus be assisting the desired penetration of the mycelium without requiring un-controlled mechanical actions unto or into the mycelium material.

It should be noted that the amount of internal process liquid will increase during the wetting process, and that mechanical assisting of penetration into the mycelium invoked by the externally provided flow may be gradually possible to a larger extent when the mycelium is softening up due to the induced water.

According to an embodiment of the invention, the liquid flow is established in proximity to at least 30-100% of the physical boundaries of the mycelium material, such as 40-100% of the physical boundaries of the mycelium material, such as 50-100% of the physical boundaries of the mycelium material, such as 60-100% of the physical boundaries of the mycelium material, such as 70-100% of the physical boundaries of the mycelium material, such 80-95% of the physical boundaries of the mycelium material.

According to an embodiment of the invention, the circulation of process liquid outside the mycelium storing volume is obtained by means of activation of one or more pumps located in the process tank and/or coupled to the process tank.

According to an embodiment of the invention, the one of more pumps are controlled by a controller.

The one or more pumps applied for generating a movement of liquid in the exterior liquid is typically coupled to a power supply and the pump is controlled by a controller. The controller may comprise one or more signal processors supplied by a power supply, and the one of more signal processors are operating according to software instructions layer in an data memory associated to the signal processer. The controller may e.g. control the pump speed and thus the output of the pump. I input variables are given by various sensors, such as flow meters and level sensors. Then information triggers the pump controller, which then adjusts the speed of the pump to the optimal. The controller is adapted to fit the intended purpose and the applied type of pump. Different type of pumps may be controlled differently and the use of sensor(s) may be different, as long as the desired process liquid flow is established.

According to an embodiment of the invention, the actively established liquid flow is obtained by means of coordinated activation by the controller of a two or more pumps located in the process tank and/or coupled to the process tank to establish a flow pattern in the exterior process liquid.

A coordinated activation may include, considering the locations of the pumps involved, an activation of the pumps so that the pumps pump in the same direction and/or opposite directions. The pumps or some/one of the of the pumps may also be reversed during the pumping process in order to modify the flow pattern in the process tank.

According to an embodiment of the invention, the process tank comprises one or more process liquid inputs and one or more process liquid outputs.

According to an embodiment of the invention, the process tank comprises one or more process liquid inputs and one or more process liquid outputs and wherein the liquid input(s) and or the output(s) are coupled to a pump, the pump establishing a flow between said liquid inlet(s) and outlet(s).

According to an embodiment of the invention, the flow into the mycelium material is at least partly established by a mechanical and automatically controlled compression and de-compression in at least one compression cycle.

In the present context, a compression cycle involves that the mycelium material, when being submerged in process liquid, are compressed and decompressed in a cycle allowing the process liquid and optional air/gas contained within the outer boundaries of the mycelium material to flow out of the mycelium material when compressed and soaking liquid into the mycelium material during de-compression, when the mycelium material returns back to/closer back to unpressed state due to mechanical forces in the mycelium. The compression and decompression of the mycelium material is performed to obtain at least a well-defined thickness when the mycelium is compressed to its most compressed state.

In the present context, well-defined has the meaning that the compression is performed in an non-arbitrary way by the controller and preferably that the exact compression is predefined to a degree that algorithms executed by the controller determines the exact level of maximum compression, typically defined by thickness of the mycelium material.

According to an embodiment of the invention, mechanical compression and decompression is performed at least during a part of the wetting process.

According to an embodiment of the invention, it is possible to compress and decompress the mycelium material at least one time during the wetting process, and thereby obtain that gas is “squeezed out” of the mycelium material during the compression part of the cycle and that water is soaked into the mycelium material during de-compression.

In the present context, de-compression may simply mean that the compression force is terminated, e.g. by lifting a compression plate (perforated) from the mycelium and then the mechanical forces of the mycelium material will tend to expand the mycelium to (or closer to) the dimension of the mycelium material prior to compression. In other words, the de-compression may as such be implemented as a passive step as a simple reaction to termination of the compression pressure on the mycelium material.

A number of cycles of compression may be performed, preferably automatically, during the wetting process. The number of cycles may increase during the wetting process and the cycle time period may vary.

According to an embodiment of the invention, the mechanical compression of the compression/de-compression cycle(s) performed on the mycelium material is increased during the wetting process.

The mechanical compression may be increased to obtain that the thickness of the mycelium material during e.g. a wetting process at the end of each compression of a compression/de-compression cycle is becoming gradually thinner when the compression of a compression and de-compression cycle is at its maximum. The thickness may or may not return to the thickness of the mycelium sheet prior to compression.

According to an embodiment of the invention, the system comprises a mycelium support base and a corresponding compression member arranged within the volume of the process tank,

According to an embodiment of the invention, the mycelium support base and the compression member being configured for mutual displacement, controlled by an associated controller, towards and away from said mycelium support base in at least one compression and de-compression cycle, wherein the mutual displacement is obtained by means of at least one actuator controlled by the associated controller.

The process tank should be designed so that it is possible to position mycelium in the tank on the mycelium support base prior to initiating e.g. a wetting process, plasticizing process, a tanning process and or a dyeing process.

The present mycelium processing system provides a way of effectively processing a mycelium whether it is applied for a wetting process, plasticizing process, a tanning process and/or a dyeing process in that a liquid flow may be promoted into and through mycelium sheet(s), when positioned on the mycelium support base(s) and the mycelium sheet(s) are subjected to compression by means of a cooperating support base and compression member.

According to an embodiment of the invention, the mycelium support base has a support base surface.

According to an embodiment of the invention, the compression member has a compression member surface.

The invention further relates to an industrial process of processing a mycelium material, preferably into a non-woven fabric, the process including the steps of providing a mycelium material, positioning the mycelium material in a process tank, inputting process liquid into the process tank, a part of the process liquid in the tank forming exterior process liquid being located outside the physical boundaries of the mycelium material, actively establishing a liquid flow in the exterior process liquid, wherein the liquid flow is established in proximity to at least a part of the physical boundaries of the mycelium material.

According to an embodiment of the invention, the mycelium material is subsequently compressed into a non-woven fabric.

In the present context establishing of a liquid flow in the exterior process liquid means a process where as little as possible mechanical stress is induced to the mycelium material during wetting of chemical treatment. This means in practice that uncontrolled mechanical stress should be avoided as such stress may result in damaging of the mycelium fibers.

As for the process liquid and the definitions regarding to location of this process liquid, it is noted that the location of process liquid during the active induction of flow in the exterior liquid is changing all the time, both due to the active establishment of liquid flow in the exterior process liquid but also due to passive movement of process liquid.

The exact location and the amount process liquid is therefore intentionally changing during wetting, de-wetting, rinsing and/or chemical treatment of the mycelium unless a desired equilibrium has been obtained.

The inventive process is e.g. beneficial for the purpose of obtaining a gently way of transporting process liquid into the mycelium material to obtain the desired penetration of process liquid.

The inventive process benefits in that it is possible to avoid a tumbling process which is typically applied in a tanning process. A tumbling process represents a type of mechanical stress which in uncontrolled and the process may very likely damage the mycelium fibres. It is thus preferred that any mechanical process applied for assisting the present way of transporting process liquid into/through the mycelium fibres is carefully and predictably controlled so as to ensure that optional damage to the mycelium fibres are avoided or kept at a low level.

According to an embodiment of the invention, the inducement of active flow over a duration of time, will promote penetration of liquid into the mycelium and thereby promote an efficient plasticising, whether or not the active flow of process liquid is primarily water during an initial wetting process prior to plasticising of mycelium material.

The active flow established is in particular facilitating a uniform penetration of process liquid into the mycelium material and thereby promoting a uniform strength throughout the cross-section of a final fabric. This is in particular relevant in the field of fabrics based on mycelium material, as the starting material is brittle and susceptible to breaking of not properly plasticized.

### SEPARATE WETTING PROCESS PRIOR TO TANNING PROCESS

According to an embodiment of the invention, the mycelium is subjected to a wetting process step prior to a plasticizing process step and/or a tanning process.

According to an embodiment of the invention, the wetting process step includes subjecting the mycelium material to process liquid in the form of water prior to the tanning process of the mycelium.

According to an embodiment of the invention, the mycelium material is submerged in the process liquid at least a part of the duration of the wetting process step.

According to an embodiment of the invention, the mycelium material is submerged in the process liquid at least 50% of the duration of the wetting process step.

In an embodiment of the invention, the mycelium material is submerged in the process liquid at least 60% of the duration of the wetting process step.

In an embodiment of the invention, the mycelium material is submerged in the process liquid at least 70% of the duration of the wetting process step.

In an embodiment of the invention, the mycelium material is submerged in the process liquid at least 80% of the duration of the wetting process step.

In an embodiment of the invention, the mycelium material is submerged in the process liquid at least 90% of the duration of the wetting process step.

In an embodiment of the invention, the mycelium material is submerged in the process liquid at least 95% of the duration of the wetting process step.

In an embodiment of the invention, the mycelium material is submerged in the process liquid at least 98% of the duration of the wetting process step.

In an embodiment of the invention, the mycelium material is submerged in the process liquid the complete duration of the wetting process step.

According to an embodiment of the invention, the wetting process step has a duration of 1 to 72 hours, such as 2 to 48 hours, such as 3 to 24 hours, such as 4 to 12 hours.

In the present context, duration of a wetting process addresses the part of the process prior to plasticizing process steps, as the wetting process is primarily focused on obtaining a desired humidity (wetting) throughout the mycelium material prior to the subsequent plasticizing, as “total” wetting facilitates not only a good penetration of plasticizing agent/tanning agent subsequent to the wetting, but also facilitates that the proteins bindings sites are properly prepared when protein targeting agents are added.

It has thus been established during realistic test that a failure to perform an effective pre-wetting of the mycelium material may have an effect on the final strength properties of the processed mycelium material. If the mycelium material is finally processed/pressed into a fabric, e.g. applied in footwear, this lack of pre-wetting may materialize in that that fibres of the mycelium is not properly plasticized and consequently breaks during stress/use.

According to an embodiment of the invention, the wetting process step has a duration of 2 to 48 hours, such as 3 to 40 hours.

According to an embodiment of the invention, the wetting process is performed without performing mechanical compression on the mycelium material.

According to an embodiment of the invention, process liquid applied in the wetting process step includes water and a surfactant.

The surfactant is beneficial to the process as the wetting and the tanning process of the mycelium is intended to be as gentle as possible and thereby avoiding a tumbling process which is typically applied in a tanning process. A tumbling process represents a type of mechanical stress which in uncontrolled and the process may very likely damage the mycelium fibres. It is thus preferred that any mechanical process applied for assisting the present way of transporting process liquid into/through the mycelium fibres is carefully and predictably controlled so as to ensure that optional damage to the mycelium fibres are avoided or kept at a low level.

Surfactants are compounds that lower the surface tension (or interfacial tension) between two liquids, between a gas and a liquid, or between a liquid and a solid. In the present context, the surface tension between a liquid, typically water during the wetting process, is a particular concern in order to speed up the wetting process with little as possible use of mechanical stress to the mycelium.

Surfactants are usually organic compounds that are amphiphilic, meaning they contain both hydrophobic groups (their tails) and hydrophilic groups (their heads). Therefore, a surfactant typically contains both a water-insoluble (or oil-soluble) component and a water-soluble component. Surfactants will diffuse in water and adsorb at interfaces between the water (e.g. water applied during the wetting process) and the mycelium material.

Applicable surfactants include e.g. Invaderm LU, soap or other surfactant suitable for lowering the surface tension between the water/process liquid and the mycelium.

In an embodiment, the surfactant is only added during the wetting process in a separate process step of the tanning process and prior to the tanning process.

The wetting process may by performed by process liquid of water with 1 to 20% by weight of surfactant, such as 2 to 15% by weight of surfactant.'

According to an embodiment of the invention, the wetting process is followed by a subsequent tanning process step.

A tanning process in the present context involves plastification of the mycelium material and this is done by the application of plastification agents (plasticizer), such as tanning agents known from the leather industry.

According to an embodiment of the invention, the mycelium is submerged in the process liquid at least a part of the duration of the tanning process.

Wherein the mycelium is submerged in the process liquid at least 50 of the duration of the tanning process step and wherein the tanning process step has a duration of between 1 hour and 72 hours, such as between 2 hours and 48 hours.

According to an embodiment of the invention, the mycelium material comprises between 50 and 90% water by weight prior to the wetting process.

According to an embodiment of the invention, the mycelium material comprises between 50 and 80% water by weight prior to the wetting process.

According to an embodiment of the invention, the mycelium material comprises between 10 and 50% water by weight prior to the wetting process.

According to an embodiment of the invention, the mycelium is located in a submerged stage in process liquid in at least one process tank during the process.

According to an embodiment of the invention, the mycelium is located in a submerged stage in process liquid in at least one process tank during the process, in an initial part of the process, the process tank being a wetting tank, and in at subsequent part of the process the process tank being a plasticizing tank, a tanning tank, and/or a dyeing tank.

According to an embodiment of the invention, the mycelium material, such as one or more mycelium sheets, is/are oriented in the horizontal direction during the wetting process, the plasticizing process, the tanning process and/or the dying process.

According to an embodiment of the invention, the system comprises a mycelium support base and a corresponding compression member arranged within the volume of the process tank, the mycelium support base and the compression member being configured for mutual displacement, controlled by an associated controller, towards and away from said mycelium support base in at least one compression and de-compression cycle, wherein the mutual displacement is obtained by means of at least one actuator controlled by the associated controller.

The process tank should be designed so that it is possible to position mycelium in the tank on the mycelium support base prior to initiating e.g. a wetting process, plasticizing process, a tanning process and or a dyeing process.

The present mycelium processing system provides a way of effectively processing a mycelium whether it is applied for a wetting process, plasticizing process, a tanning process and/or a dyeing process in that a liquid flow may be promoted into and through mycelium sheet(s), when positioned on the mycelium support base(s) and the mycelium sheet(s) are subjected to compression by means of a cooperating support base and compression member.

According to an embodiment of the invention, the mycelium support base has a support base surface.

According to an embodiment of the invention, the compression member has a compression member surface.

According to an embodiment of the invention, the processed mycelium material is compressed into a final non-woven fabric subsequent to the wetting process and the plasticizing process.

According to an embodiment of the invention, the processed mycelium material is compressed into a final non-woven fabric subsequent to the wetting process step, the plasticizing process step and the dyeing process step.

According to an embodiment of the invention, the flow into the mycelium material is at least partly established by a mechanical and automatically controlled compression and de-compression in at least one compression cycle.

In the present context, a compression cycle involves that the mycelium material, when being submerged in process liquid, are compressed and decompressed in a cycle allowing the process liquid and optional air/gas contained within the outer boundaries of the mycelium material to flow out of the mycelium material when compressed and soaking liquid into the mycelium material during de-compression, when the mycelium material returns back to/closer back to unpressed state due to mechanical forces in the mycelium. The compression and decompression of the mycelium material is performed to obtain at least a well-defined thickness when the mycelium is compressed to its most compressed state.

In the present context, well-defined has the meaning that the compression is performed in an non-arbitrary way by the controller and preferably that the exact compression is predefined to a degree that algorithms executed by the controller determines the exact level of maximum compression, typically defined by thickness of the mycelium material.

According to an embodiment of the invention, mechanical compression and decompression is performed at least during a part of the wetting process.

According to an embodiment of the invention, it is possible to compress and decompress the mycelium material at least one time during the wetting process, and thereby obtain that gas is “squeezed out” of the mycelium material during the compression part of the cycle and that water is soaked into the mycelium material during de-compression.

In the present context, de-compression may simply mean that the compression force is terminated, e.g. by lifting a compression plate (perforated) from the mycelium and then the mechanical forces of the mycelium material will tend to expand the mycelium to (or closer to) the dimension of the mycelium material prior to compression. In other words, the de-compression may as such be implemented as a passive step as a simple reaction to termination of the compression pressure on the mycelium material.

A number of cycles of compression may be performed, preferably automatically, during the wetting process. The number of cycles may increase during the wetting process and the cycle time period may vary.

According to an embodiment of the invention, the mechanical compression of the compression/de-compression cycle(s) performed on the mycelium material is increased during the wetting process.

The mechanical compression may be increased to obtain that the thickness of the mycelium material during e.g. a wetting process at the end of each compression of a compression/de-compression cycle is becoming gradually thinner when the compression of a compression and de-compression cycle is at its maximum. The thickness may or may not return to the thickness of the mycelium sheet prior to compression.

According to an embodiment of the invention, the inputting of process liquid is performed prior to, subsequent to and/or at the same time as the mycelium material is positioned in the process tank.

According to an embodiment of the invention, the inputting and/or a draining of a process tank is performed by means of a at least one pump.

### THE DRAWINGS

Various embodiments of the invention will in the following be described with reference to the drawings where

FIGS. 1 and 1b illustrate a wetting process of a fungus material,

FIG. 2a-c illustrate an embodiment of the invention with internal flow inducers,

FIG. 3 illustrates an embodiment where process liquid flow is obtained by means of external flow inducers,

FIGS. 4a and 4b illustrate an embodiment of the invention with external flow inducers,

FIGS. 5a to 5e illustrate an operation procedure in a process tank,

FIG. 6 illustrate a further embodiment of the invention configured with a further process liquid tank,

FIG. 7 illustrates a configuration of a pump and associated control circuitry,

FIG. 8a-c illustrate different implementations of a mycelium support base and corresponding compression member within the scope of the invention,

FIGS. 9a and 9b illustrate a mycelium panel in a non-compressed state and a compressed state,

FIG. 10a-c illustrate a compression and de-compression cycle,

FIGS. 11a and 11b illustrate a version of stacked pairs of corresponding mycelium support bases and compression members in an advantageous embodiment of the invention,

FIGS. 12a to 13b illustrate different operational patterns of compression and de-compression cycles in the time domain within the scope of the invention,

FIG. 14a-14e illustrate different steps of process step within the scope of the invention,

FIG. 15 illustrate an embodiment of the invention applying both mechanical compression and establishment of liquid flow in the exterior process liquid,

FIG. 16 illustrates a flow chart of selected mycelium processing steps within scope of the invention,

FIG. 17 illustrates a serial implementation of the above illustrated process steps,

FIG. 18 illustrates the principle of a tank operation which may perform two or more of the mycelium processing steps of FIG. 16 in one process tank

### DETAILED DESCRIPTION

### Mycelium

Mycelium is the vegetative part of a fungus or fungus-like bacterial colony, consisting of a mass of branching, thread-like hyphae. The mass of hyphae is sometimes called shiro, especially within the fairy ring fungi. Fungal colonies composed of mycelium are found in and on soil and many other substrates. A typical single spore germinates into a monokaryotic mycelium, which cannot reproduce sexually; when two compatible monokaryotic mycelia join and form a dikaryotic mycelium, that mycelium may form fruiting bodies such as mushrooms. A mycelium may be minute, forming a colony that is too small to see, or may grow to span thousands of acres as in Armillaria.

Through the mycelium, a fungus absorbs nutrients from its environment. It does this in a two-stage process. First, the hyphae secrete enzymes onto or into the food source, which break down biological polymers into smaller units such as monomers. These monomers are then absorbed into the mycelium by facilitated diffusion and active transport.

Mycelia are vital in terrestrial and aquatic ecosystems for their role in the decomposition of plant material. They contribute to the organic fraction of soil, and their growth releases carbon dioxide back into the atmosphere (see carbon cycle). Ectomycorrhizal extramatrical mycelium, as well as the mycelium of arbuscular mycorrhizal fungi, increase the efficiency of water and nutrient absorption of most plants and confers resistance to some plant pathogens. Mycelium is an important food source for many soil invertebrates. They are vital to agriculture and are important to almost all species of plants many species co-evolving with the fungi. Mycelium is a primary factor in a plant's health, nutrient intake, and growth, with mycelium being a major factor to plant fitness.

The mycelium process steps addressed in the present application may be understood as part of the mycelium processing steps which happens after the mycelium has been separated from the growth bed and when the living organisms in the fungus has preferably been killed.

The mycelium material may, at the stage be carved out to have a certain dimension—or it may have been grown into the intended dimension.

### Growth Medium

Growth medium may vary depending on type of fungus and the desired end properties, applied growth process, applications, etc.

### Dimension

The mycelium material applied in the present context, should have a 3-dimensional organic structure, where the structure after compression or collapsing may be brought into a fabric, which may e.g. have characteristics resembling e.g. leather in terms of how the fabric is perceived and experienced by a user.

The mycelium material should, preferably prior to wetting, tanning, dyeing and/or compression be provided in dimensions suitably for providing e.g. wearables, upholstery for furniture in homes, cars, etc.

An applicable dimension of such a mycelium material is e.g. 2 meters×12 meters×10 cm.

A mycelium starting material, which may also be referred to as a mycelium panel and may have a width and length of 1 to 50 meters in the horizontal directions and a thickness of between 5 cm to e.g. 1 m. The thickness will rely very much on the intended strength of the final fabric to be produced and the weight density of the fungus starting material.

The mycelium material may be grown into the intended and desired dimensions and/or may be carved out from a mycelium.

The mycelium starting material will during the subsequent process steps be subjected to necessary processing and then, when ready, by compressed into a mycelium sheet which may form the final non-woven fabric or preferably receive post compression treatment into the final non-woven mycelium fabric.

It should be noted that the compression applied for bringing the mycelium panel into its final shape/thickness is a compression which is different from the compression applied during the optionally applied compression and de-compression cycle applied for inducing flow of process liquid into the mycelium panel.

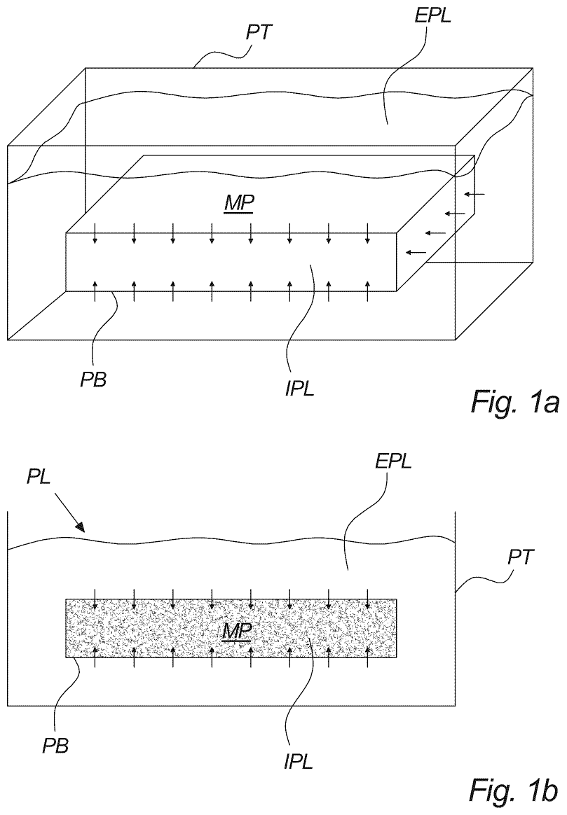

FIGS. 1 and 1b illustrate principles of a mycelium processing step within the scope of the invention. A mycelium panel MP is positioned in process liquid PL contained in a process tank PT. The process liquid may have different compositions depending on which type of mycelium processing step is being performed. The illustrated mycelium has outer physical boundaries PB.

Different mycelium processing steps will be explained below. It is nevertheless noted that an initial processing step, a wetting process step, may apply process liquid in the form of water with no added compounds or only one or few simple compounds, e.g. surfactant(s).

During a wetting process step, a main object is to make process liquid flow into the mycelium panel and saturate (or nearly saturate) the mycelium with water and optional further compounds prior to further process steps, such as a plastification process step, a tanning process step and a dyeing process step. In a wetting process step, it is preferred that such further optional compounds are not selected from the group of plasticizers, tanning agents and fatliquors as it may be advantageous that the wetting process step saturates the mycelium panel(s) prior to the subsequent plastification, tanning, dyeing, fatliquoring, as a wetting of the mycelium panel prior to addition of such compounds may facilitate in a more uniform and predictable plasticizing of the mycelium panel.

An initial wetting process and the process steps subsequent to the wetting process step relies on a sufficient penetration of process assisting liquid into the mycelium panel, and this may be applied in different ways within the scope of the of the invention.

A challenge in relation to the task of penetrating process liquid into the mycelium panel is that such a process may take a very long time unless the penetration process is assisted. It is in particular desirable to assist the initial wetting process, irrespective what the initial content of moisture is in mycelium panel—as long as the mycelium panel is below saturation.

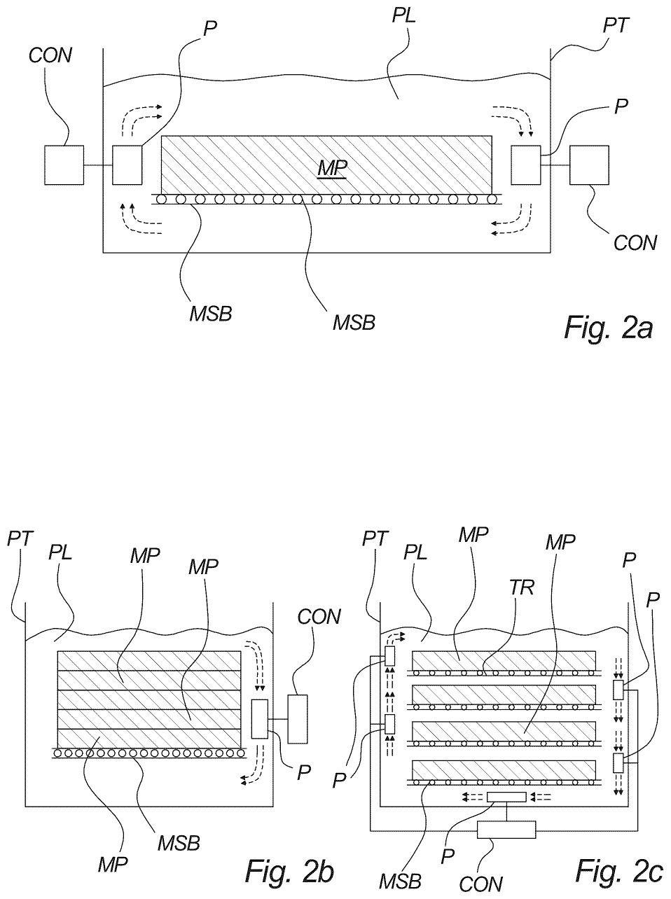

FIG. 2a-c illustrate a cross-section of an embodiment of the invention with internal flow inducers. The flow inducers, e.g. in the form of pumps P, are located within a process tank PT. A mycelium panel is submerged into process liquid. Depending on the level of saturation, a part of the process liquid PL will be located within the physical boundaries of the mycelium panel MP. The part of the process liquid will be referred to as internal processing liquid IPL and the part of the process liquid being outside the physical boundaries of the mycelium panel is referred to as exterior process liquid EPL. If the process is a wetting process step, the initial moisture content may be as low as below 10% of the moisture level at saturation, or preferably between 40 to 80%.

It is noted that part of the moisture IPL “trapped” within the physical boundaries PB of the mycelium panel may comprise both moisture trapped between the mycelium fibers/hyphae and also the moisture trapped within the mycelium. The aim of an initial wetting process is to penetrate as much process liquid as possible into both the mycelium negative space and the mycelium positive space to promote an efficient interaction with subsequently applied processing agents, such as plasticizers, tannins, dyes etc.

It should be noted that mycelium panels may have absorption abilities which depends on the type of growth medium.

The mycelium process tank PT includes a mycelium support base MSB upon which a mycelium panel MP is positioned submerged in process liquid PL contained in the process tank PT.

The mycelium support base MSB is supported mechanically directly or indirectly by the process tank by means of an attachment arrangement (not shown).

Two flow inducers, here two pumps P are located in the tank volume and they are controlled by respective controllers CON (the controllers may be embodied to be parts of the same co-functioning controller)

The pumps are controlled to provide a circulation of exterior process liquid EPL thereby to obtain that process liquid are inducing process flows which may invoke or assist an accelerated penetration of process liquid into the mycelium panel when compared to a process where the mycelium panel is simply submerged into a process liquid for passive soaking of the process liquid.

The pumps P may be controlled to provide the best suited liquid flow e.g. by synchronizing one pump P with the other as indicated in FIG. 2a, where the right pump induces process liquid flow from the top of the tank towards the bottom of the tank and where the left pump induces flow from the bottom of the tank towards the top of the tank, thereby promoting a circular flow pattern.

Other patterns may be applied within the scope of the invention, and the pumps may also be controlled over time, e.g. by reversing the direction of flow from time to time or by modifying the flow speed of one or more pumps.

FIG. 2b illustrates a variant of FIG. 2a, where a number of mycelium panels are stacked directly upon each other and where a flow of process liquid is obtained by a pump operating within the tank volume of the process tank PT and being controlled by a respective controller CON.

FIG. 2c illustrates a further embodiment within the scope of the invention where a number of mycelium panels are stacked on respective mycelium support bases MSB allowing flow between the stacked mycelium panels MP.

A number of pumps controlled by a controller (not shown) establishes a process liquid flow, thereby promoting penetration of process liquid into the mycelium panels without direct mechanical uncontrolled/unpredictable manipulation of the mycelium panel.

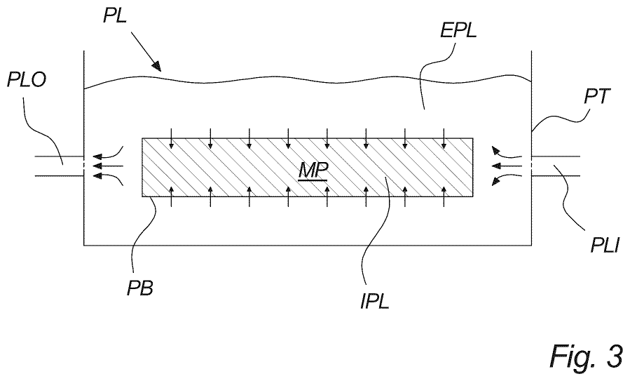

FIG. 3 illustrate a further way of obtained flow in the process liquid, namely by flow inducers (not shown) operating exterior to the process tank and allowing process liquid into the process tank PT through at process liquid inlet PLI and by actively sucking process liquid out of a process liquid outlet PLO by means of a flow inducer (not shown), e.g. a pump.

A mycelium panel MP is located on a mycelium support base MSB (not shown). Further mycelium panels and mycelium support bases MSB may be applied in the present embodiment although only one of each is indicated.

Again the active establishment of a liquid flow exterior to the physical boundaries of the mycelium panels promotes penetration of process liquid into the mycelium panel.

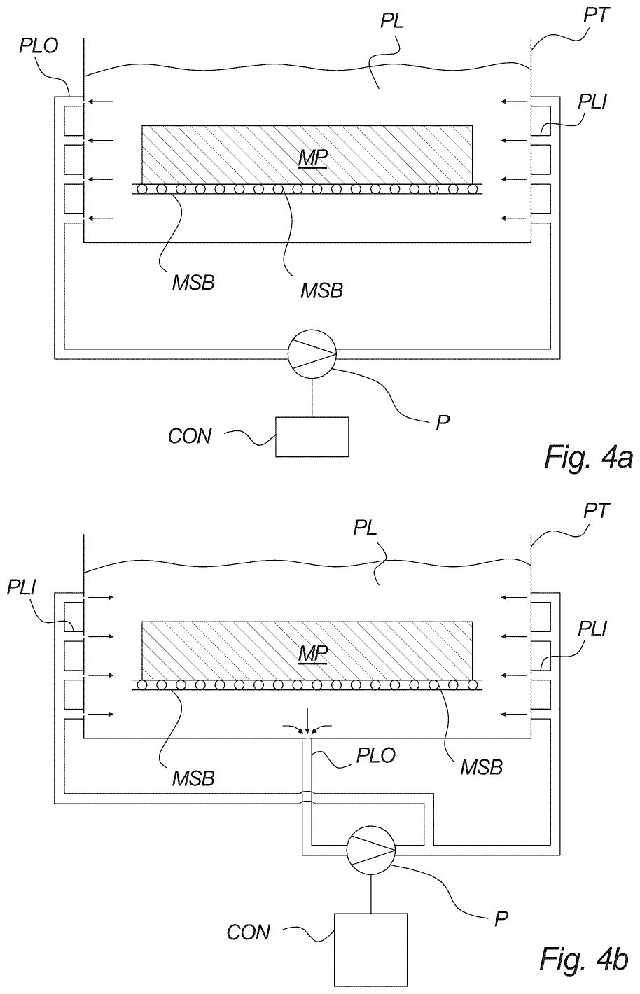

FIG. 4a illustrates an embodiment of the invention with external flow inducers, where a pump P is controlled by a controller CON to establish a circulation of process liquid between process liquid outlets PLO and process liquid inlets PLI and thereby creating a flow between the process liquid outputs PLO and the process liquid outputs within the process tank.

A mycelium panel MP is located on a mycelium support base MSB. Further mycelium panels and mycelium support bases MSB may be applied in the present embodiment although only one of each is indicated.

Again the active establishment of a liquid flow exterior to the physical boundaries of the mycelium panels promotes penetration of process liquid into the mycelium panel.

FIG. 4b illustrates are further variant of the embodiment of FIG. 4a, now arranged with a further process liquid output, thereby modifying the process liquid flow in the process tank PT when compared to the liquid flow pattern of FIG. 4a.

The further process liquid outlet is here located a the bottom of the tank.

Any suitable desired number of process liquid inputs and process liquid outputs may be applied within the scope of the invention the localization of the process liquid output(s) and input(s) may be designed to obtain the desired liquid flow path. This a general principle of all embodiments applying flow inducers external to the process tank indicated or explained in the present patent application.

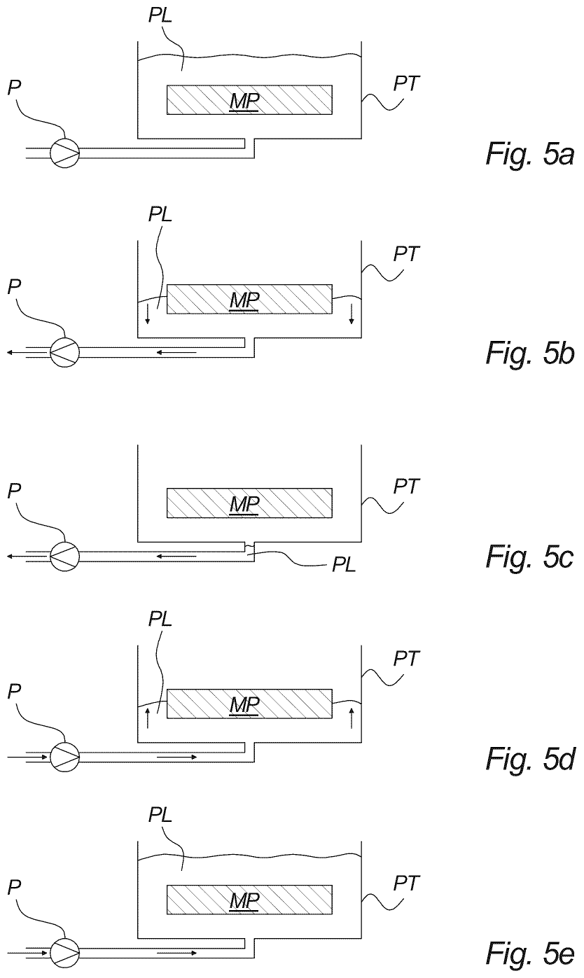

FIGS. 5a to 5e illustrate a filling and emptying procedure applied in a process tank PT within the scope of the invention. The illustrated figures are shown to illustrated that different mycelium processing steps may be performed in the same process tank or process liquid may be exchanged when a mycelium panels is located within the process tank (several mycelium panels may be located in the process tank even if only one mycelium panel is indicated)

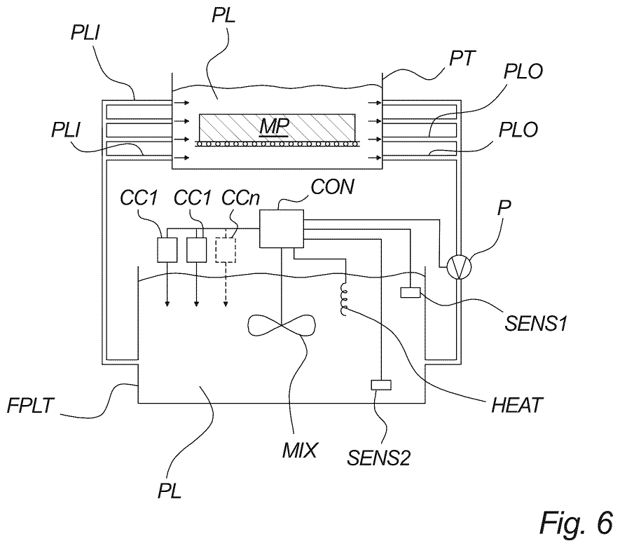

FIG. 6 illustrates a further embodiment of the invention configured with a further process liquid tank FPLT. The further process liquid tank FPLT is fluidly coupled with a process tank, here comprising process liquid and a pump is controlled the liquid flow of process liquid PL into and out of the process tank PT via multiple process fluid inlets PLI and process fluid outlets PLO.

A mycelium panel MP is located on a mycelium support base MSB. Further mycelium panels and mycelium support bases MSB may be applied in the present embodiment although only one of each is indicated.

Furthermore, the fluid outlets PLO are fluidly coupled via a pump P controlled by a controller CON to the further process liquid tank FPLT, thereby allowing the process liquid may be pumped from the process tank into the further process liquid tank FPLT

Also, the fluid liquid inlets PLI are fluidly coupled to the further process liquid tank FPLT so as to guide process liquid from the further process liquid tank FPLT into the process tank in response to the pump pumping process liquid out of the process tank PT.

Evidently, more pumps may be applied to obtain the desired circulation, e.g. one pump as illustrated, associated with process liquid outlets PLO and a further pump (not shown) optionally be applied to actively pump process liquid from the further process liquid tank into the process tank via the process liquid outlets PLO under the control of the illustrated controller or a dedicated controller (not shown)

The further process liquid tank FPLT contains process liquid PL which may vary slightly in concentration of process compounds from the concentration of the same compound in the process liquid contained in the process tank PT, but the difference is kept low by circulation of process liquid, not necessarily invoked during the total duration of the process step in question, but at least the circulation is maintained so that the concentration of relevant process compounds in process liquid in the process tank and process liquid in the further process liquid tank are converging towards the same level of concentration. This is highly advantageous, as addition of process compound to the liquid may be performed external to the process tank and the circulation may be controlled to obtain a relatively constant and uniform concentration of compounds added to the process liquid in the process tank, where it is desired that the concentration is within a certain range, to ensure that the processing of the mycelium panel(s) is performed according to the intended plan.

The illustrated further process liquid tank is moreover arranged with a number of injecting containers, compound containers CC1, CC2, CC3, . . . ,CCn applied for injection of respective chemical compounds and/or other relevant materials into the further process liquid tank FPLT to obtain or maintain a certain desired compound concentration over time. The dispensing compound container CC1, CC2, CC3 and CCn are under the control of the controller CON. Evidently, the injection may be performed in several different ways within the scope of the invention, e.g. by premixing the compound with water prior to injection. The injection may also be performed by e.g. a number of distributed inlets to promote an improved mixing with the process liquid. Moreover the further process liquid tank may comprise a mixer MIX controlled by the controller to obtain a desired agitation of the process liquid and a uniformity of compound concentration in the process liquid. Moreover, the process tank may comprise one or more sensors measuring e.g. compound concentrations or process liquid temperature. Any suitable numbers of sensors may be applied in the further liquid process tank FPLT and further non-shown sensor(s) may be applied in the process tank PT in order to assist the controlling of the compound concentrations, compound temperature(s), flow velocity, etc. of the mycelium processing system.

Furthermore, the further liquid process tank, the process tank and/or the liquid conduits connecting the two may be fitted a heater controlled by the controller CON.

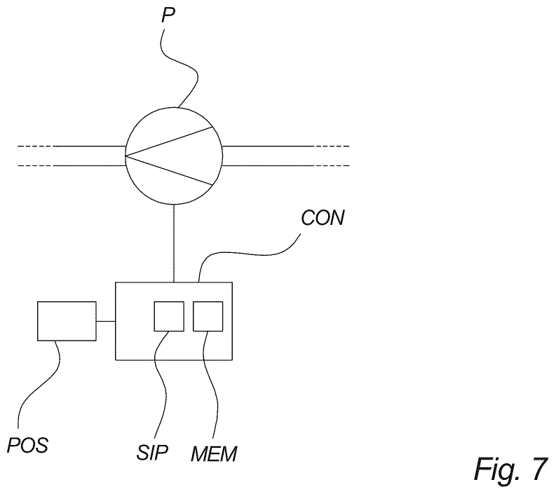

FIG. 7 illustrates the general principle of pump applied in any presently illustrated embodiments of the invention unless stated otherwise. The pump P is controlled by a controller CON. The controller may be a dedicated controller or it may be a controller also controlling other operational parameters of the inventive system.

The controller CON is operated according to executable program code stored in associated memory MEM and the code is executed by a digital processer SIP, and the controller is powered by an electrical power source POS.

The controller may be communicating with co-working controllers, not shown, and the controller may be associated to an operating system controlling the complete mycelium processing system. The operating system may further communicate with a user interface (not shown) by means of which a user may operate the mycelium processing system.

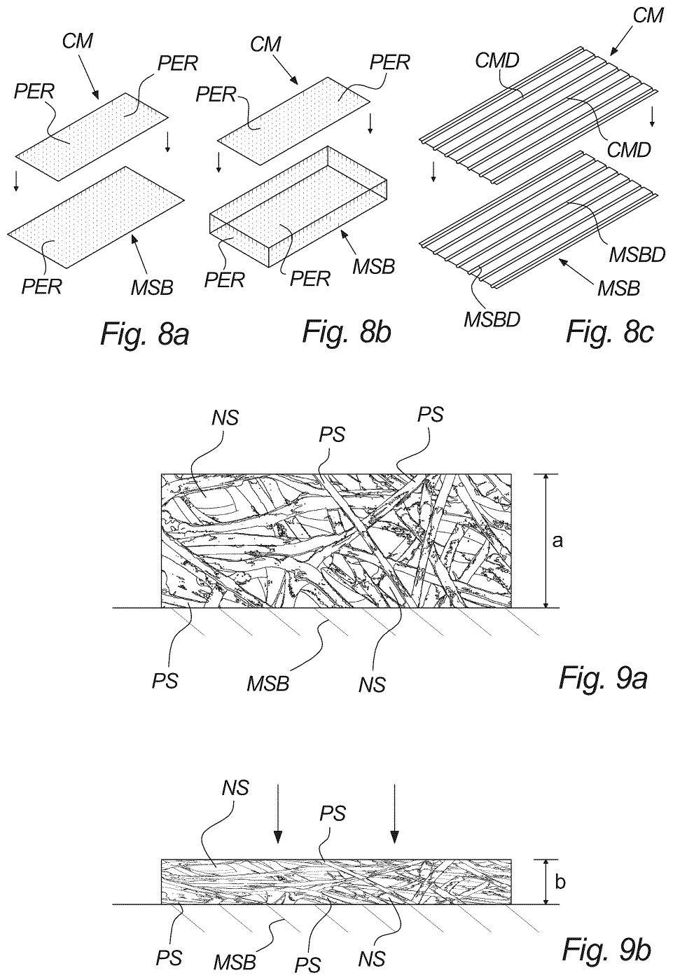

FIG. 8a-c illustrated different embodiments of mycelium support bases MSB and corresponding compression members CM. The mycelium support base of FIG. 8a and the corresponding compression member are both perforated by perforations PER to allow process liquid and gasses to pass through the perforations PER of the member and the base when the support base MSB and the compression member CM are pressed towards each other with a mycelium positioned between the support base MSB and the compression member CM.

FIG. 8b corresponds to FIG. 4a, but now the mycelium support base MSB is formed as a tray with perforations PER.

FIG. 8c illustrates the a support base MSB and a compression member CM, now formed with surfaces having longitudinally arranged support base drains MSBD and compression member drains CMD. The drain allows process liquid and gasses to be drained away from the mycelium when the support base MSB and the compression member CM are pressed towards each other with a mycelium positioned between the support base MSB and the compression member CM.

The use of the above exemplary support bases MSB and the compression members CM are illustrated in more detailed in the below embodiments.

But first, a cross view of a mycelium panel MP in a non-compressed state in FIG. 9a and in a compressed state in FIG. 9b.

The non-compressed mycelium panel has a thickness of “a” and the compressed mycelium has a thickness of “b”.

As illustrated, the mycelium defined so-called positive spaces PS where e.g. hyphae contains internal process liquid and also, the mycelium panel may comprise inter process liquid between the fungus hyphae, here illustrated as negative spaces.

A saturation of the positive spaces with water prior to plasticizing, tanning, etc. is desired, so as to obtain an improved uniformity of the desired compound within the mycelium material.

It is noted that the mycelium panel from the beginning of a wetting process is under saturated and the gasses may counteract penetration of process liquid.

In FIG. 9b gasses contained in the positive spaces between the hyphae is forced out of the mycelium panel during compression and when the mycelium panel is de-compressed it will return towards its original shape and soak process liquid into the interior of the mycelium panel.

This process may be repeated until the desired wetting has been obtained.

When the desired saturation of the mycelium panels has been obtained the mycelium panel may be subject to further process steps, such as a plasticizing step and a subsequent dyeing step.

These steps may be also be performed submerged in relevant process liquid applicable for the desired purpose, and the compounds mixing into the process liquid for the specific process steps may be actively promoted into and through the mycelium panel by the above mentioned method.

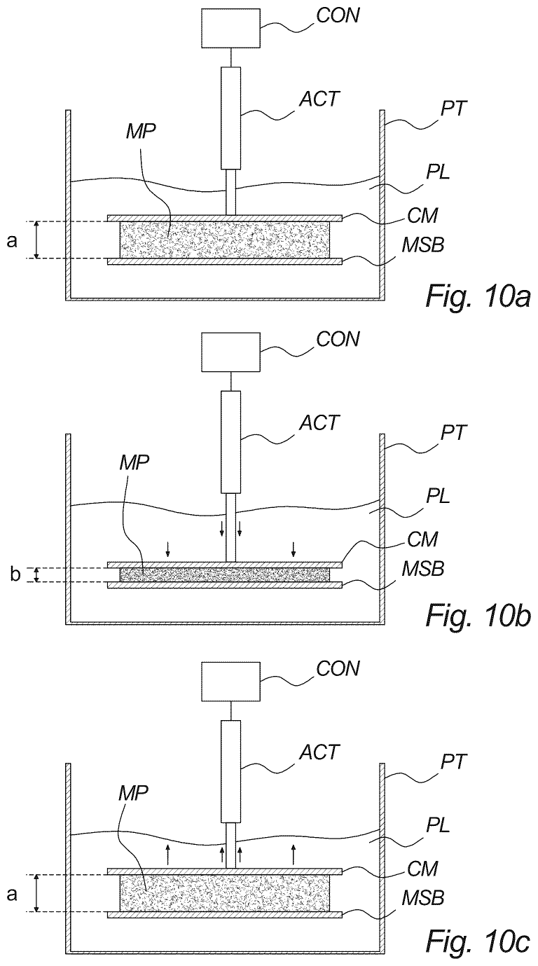

FIG. 10a-c illustrate a compression and de-compression cycle performed on a mycelium panel MP submerged in process liquid PL contained in a process tank PT.

The mycelium panel MP is positioned on a mycelium support base MSB acting as a counterpart to a compression member CM. The mycelium support base MSB and the compression member CM may e.g. be designed according to the principles illustrated in FIG. 8a-c.

A controller is controlling the movement of an actuator ACT. The actuator performs a compression cycle starting in FIG. 10a, where the thickness of the mycelium panel is “a”. In FIG. 10b the compression member has been moved relative to the mycelium support base MSB by the actuator and the thickness of the mycelium panel is “b” (it is lower). In FIG. 10c the actuator de-compress the mycelium by returning the compression member CM to its original position as illustrated in FIG. 10a and the thickness is now closer to “a” again.

This pumping process of the mycelium invokes a process liquid flow out of the mycelium panel during compression and into the mycelium panel during de-compression. A number of repeated cycles may establish and promote a flow into, out of and/or though the mycelium panel.

Moreover, the embodiment includes a flow inducer arrangement (not shown), e.g. as exemplified in any of the FIGS. 2a to 7.

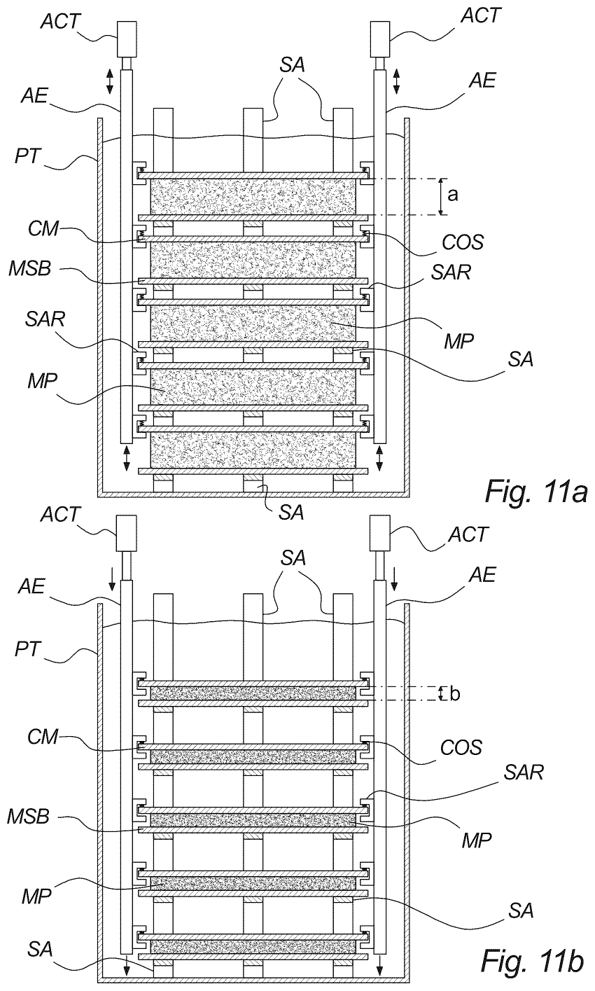

FIGS. 11a and 11b illustrate principles of a version of stacked pairs of corresponding mycelium support bases MSB and compression members CM in an advantageous embodiment of the invention.

The mycelium process system includes a process tank PT. The process tank has an inner volume to contain process liquid PL. A number of mycelium support bases are supported by the process tank PT or attached to the tank PT by means of a support assembly SA, here including a frame of vertical rods and horizontal support members supporting the mycelium support bases MSB. The support is intended to withstand relative high forces induced when a corresponding compression member CM is pressed towards the mycelium support base MSB and keep the mycelium support base MSB fixed in the vertical direction during such compression.

The compression members are suspended in horizontally oriented support assembly rails SAR fixed to actuator extenders AE preferable via compression springs COS, and the actuator extenders AE are coupled to one or more actuators ACT controlled by a controller (not shown).

The present embodiment illustrates one of several embodiments within the scope of the invention, where several mycelium panels may be compressed at the same time by one or more actuators. One actuator may thus be used to apply compression to several mycelium panels MP at the same time. Other ways of stacking co-working mycelium support bases and corresponding compression member may be applied within the scope of the invention.

In FIG. 11b the actuator ACT compresses all mycelium panels MP at the same time.

Advantageously, compression force should be monitored at all time to ensure the individual compression forces measured for each mycelium panel does not exceed a desired maximum or minimum and/or that a combined compression force measured for all mycelium panels does not exceed a desired maximum or minimum.

The actuator must be controlled to automatically compress at proper compression pressures.

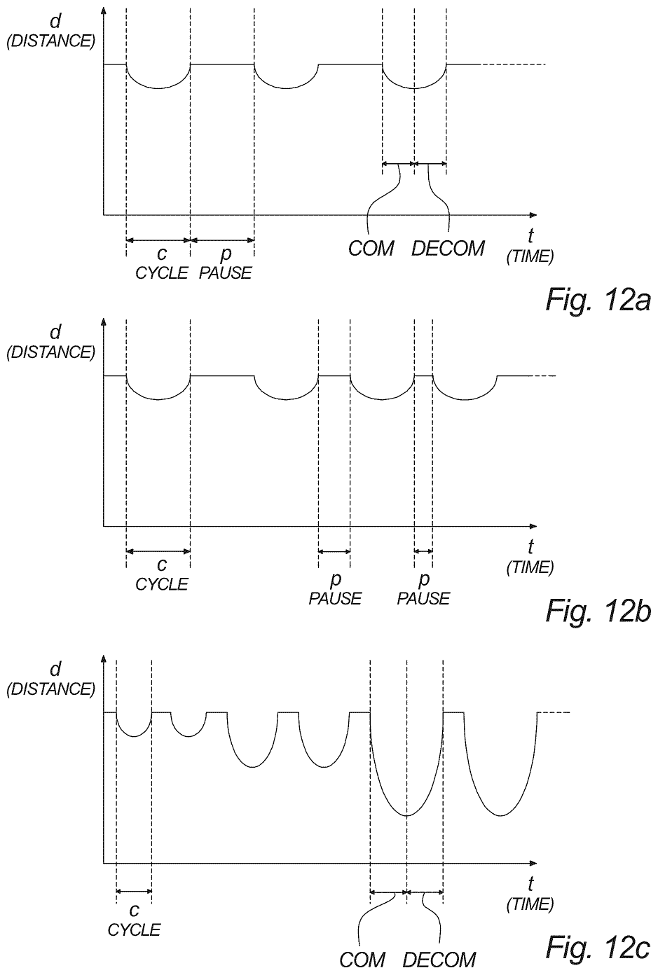

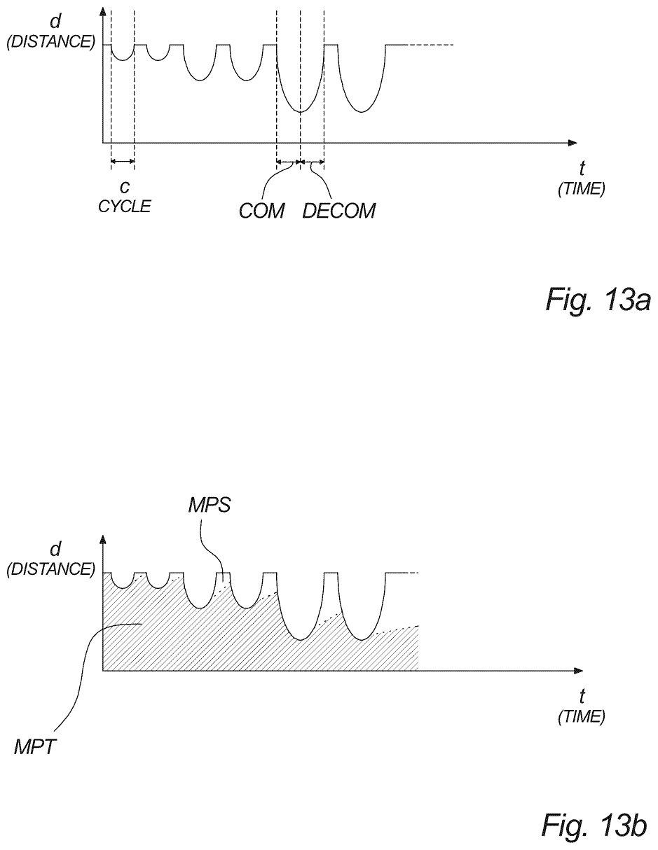

FIG. 12a to 13b illustrate different operational patterns of compression and de-compression cycles in the time domain within the scope of the invention,

The compression cycle are illustrated with time along the “TIME” axis and distance “d” between a coworking mycelium support base MSB and a respective compression member CM. It should be noted that the compression, as earlier described and indicated may be obtained through the use of a mechanically fixed mycelium support base MSB and a compression member moved by an actuator in response to control signals from an associated controller CON. Alternative configurations may be applied within the scope of the invention by the use of actuators coupled to both the mycelium support base and compression member CM.

An exemplary compression and decompression development over time is illustrated in the FIG. 12a-12c, where the distance “d” at least during compression to a large degree will directly result in a thickness of the compressed mycelium corresponding to the distance “d” whereas the thickness of the mycelium panel during de-compression will be less or the same as the distance “d”. This may depend on what type of mycelium is applied and which time development is used and it may also e.g. depend on the magnitude of the distance “d”.

A compression COM and de-compression DECOM cycle is designated as “CYCLE”. A period of no-compression is designated “PAUSE”.

It is noted that the density and number of compression and decompression cycles may be varied over time or may be the same.

The time periods may be varied. The compression time COM may be varied. The decompression period DECOM may vary and the mutual lengths of the periods may vary.

The compression and de-compression cycles are executed automatically by at least one actuator under the control of a controller CON.

It is noted that the distance between the mycelium support base MSB and a respective compression member CM may also vary over time.

This may e.g. happen during a wetting process step or some of the early process steps, where the mycelium fibers initially are somewhat fragile and where too much compression may damage the mycelium fibers irreversibly or at least damage some of the mycelium fibers irreversibly.

FIGS. 13a and 13b illustrates a further way of performing compression and de-compression cycles within the scope of the invention, where the de-compression is performed faster than the mycelium can regain or close into its original form (here: thickness of the mycelium panels subsequent to decompression and a relaxation time.

The hatched area illustrates the corresponding mycelium panel thickness MPT of the mycelium panel located between the mycelium support base MSB and a respective compression member CM. The mycelium panel thickness MPT may thus not always match the distance “d” between the mycelium support base MSB and the compression member CM which is illustrated as the mycelium panel slip MPS.

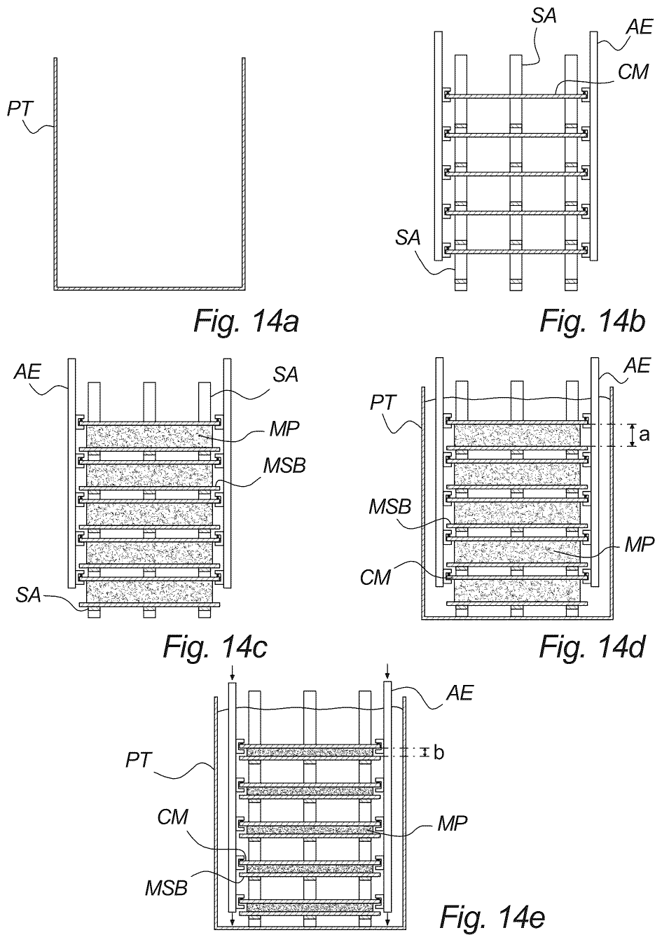

FIG. 14a-14e illustrate different process steps within the scope of the invention, when operating a mycelium process system as described in FIG. 11a-b.

FIG. 14a illustrates a process tank PT.

FIG. 14b illustrates a support assembly SA of vertical rods and horizontal support members. The support assembly SA comprises movable actuator extenders AE which a mechanically coupled to a number of compression members arranged in intervals in the vertical direction. The part of support assembly comprising the movable actuator extenders and the compression members may slide up an down independent of the stationary part of the support assembly.

In FIG. 14c mycelium panels MP are positioned within the support assembly frame SA on individual respective mycelium support members MSB. In FIG. 14d the complete support assembly SA, including mycelium panels MP has been lifted into the process tank.

As illustrated, process liquid PL has now been injected into the process tank PT, e.g. by means of a pump controlled by a controller (not shown). All the mycelium panels are now submerged in process liquid. The mycelium panel now has a thickness “a”

The actuator extenders AE have been connected with an actuator (not shown) and in FIG. 14e the mycelium panels are compressed to a thickness “b” by means of the actuator. De-compression may now be performed and further compression and de-compression cycles may be applied to establish flow into and out of the mycelium panels of relevant process liquid.

Moreover, the above embodiments of FIGS. 11a-14e include a flow inducer arrangement (not shown), e.g. as exemplified in any of the FIGS. 2a to 7.

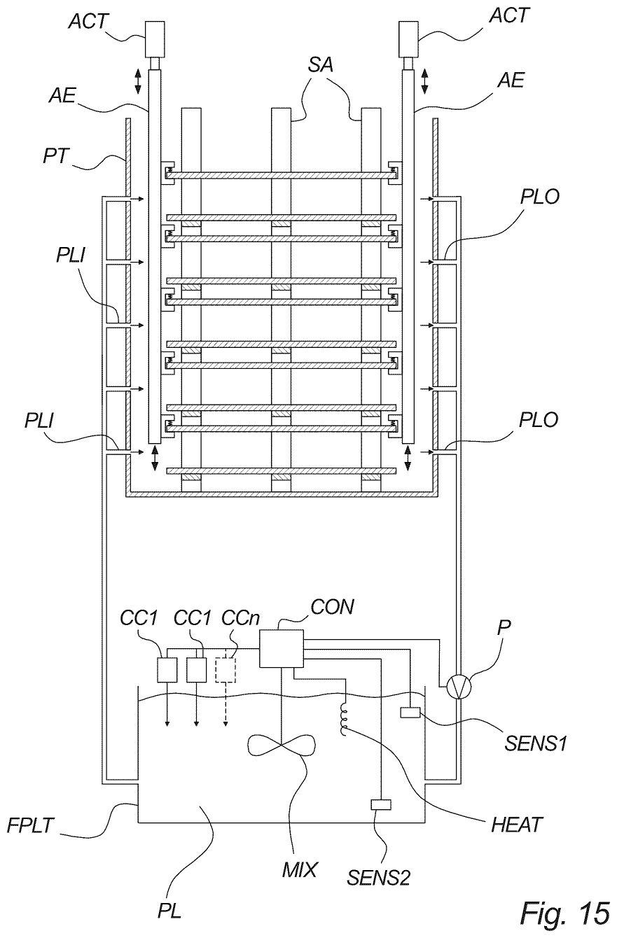

FIG. 15 illustrates an embodiment of the invention where a process tank is configured with a combination of mechanically induced compression and de-compression cycles combined with active establishment of liquid flow by flow inducers in the exterior process liquid. The embodiment broadly corresponds to a combination of FIG. 6 and FIG. 13a. Process liquid flow through the process tank and may be assisted by means of the controller CON controlled pump P when process liquid is contained in the process tank PT and in the further process tank FPT.

Process liquid flow may further be promoted by the application of one or more compression and decompression cycles applied in a relevant process step under the control of the associated controller when process liquid is contained in the process tank PT and in the further process tank FPT.

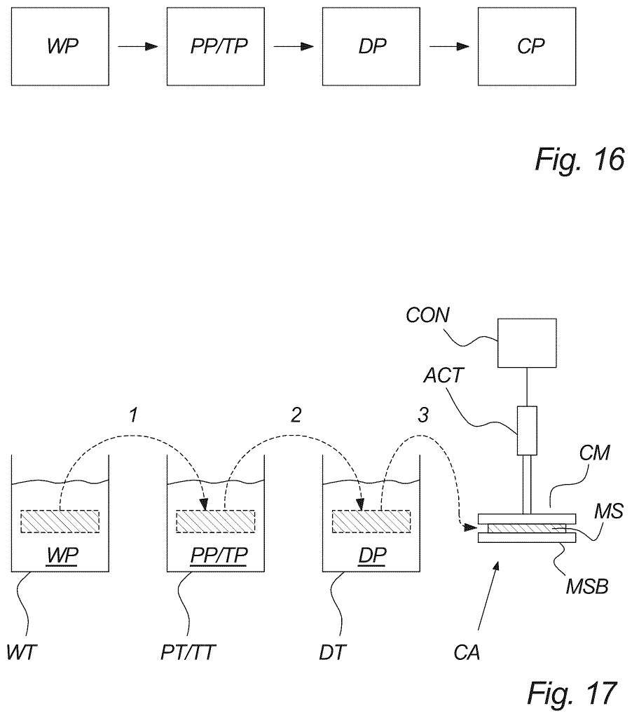

FIG. 16 illustrates a flow chart of selected mycelium processing steps within scope of the invention.

The mycelium processing steps include a wetting process step WP, a plasticizing and/or tanning process step PP/TP, a dyeing process step DP and a final compression process step CP where the mycelium panel is compressed into a mycelium sheet MS with a permanently and significantly reduces thickness. Final thickness may e.g. be between 0.5 mm and 5 mm depending on the final application. Other thickness may also apply.

Further process steps may be applied between the indicated process steps and further/supplemental processing steps may be included in the already indicated process steps.

The wetting process should preferably be performed with the primary purpose of saturating the mycelium panel with water prior to the later steps. As earlier mentioned, surfactant may be added to promote the wetting of the mycelium with water and surfactant. Plasticizing agents are preferably to be avoided at the wetting process step.

Subsequent to the process wetting step, the mycelium may be subjected to a mycelium plasticizing step (and/or a tanning step) PP/TP. The aim of this step is primarily to soften (plasticize) the mycelium fibers to increase bonding strength between the mycelium fibers.

Subsequent to the mycelium plasticizing step (and/or a tanning step) PP/TP the mycelium is subjected to a mycelium dyeing process.

Subsequently, the mycelium panel is compressed and dried into a mycelium sheet, which is now forming a non-woven mycelium fabric.

According to an embodiment of the invention, the mycelium is subjected to a wetting process step prior to a plasticizing process step and/or a tanning process.

According to an embodiment of the invention, the wetting process step includes subjecting the mycelium material to process liquid in the form of water prior to the tanning process of the mycelium.

According to an embodiment of the invention, the mycelium material is submerged in the process liquid at least a part of the duration of the wetting process step.

According to an embodiment of the invention, the mycelium material is submerged in the process liquid at least 50% of the duration of the wetting process step.

In an embodiment of the invention, the mycelium material is submerged in the process liquid at least 60% of the duration of the wetting process step.

In an embodiment of the invention, the mycelium material is submerged in the process liquid at least 70% of the duration of the wetting process step.

In an embodiment of the invention, the mycelium material is submerged in the process liquid at least 80% of the duration of the wetting process step.

In an embodiment of the invention, the mycelium material is submerged in the process liquid at least 90% of the duration of the wetting process step.

In an embodiment of the invention, the mycelium material is submerged in the process liquid at least 95% of the duration of the wetting process step.

In an embodiment of the invention, the mycelium material is submerged in the process liquid at least 98% of the duration of the wetting process step.

In an embodiment of the invention, the mycelium material is submerged in the process liquid the complete duration of the wetting process step.

According to an embodiment of the invention, the wetting process step has a duration of 1 to 72 hours.

In the present context, duration of a wetting process addresses the part of the process prior to plasticizing process steps, as the wetting process is primarily focused on obtaining a desired humidity (wetting) throughout the mycelium material prior to the subsequent plasticizing, as “total” wetting facilitates not only a good penetration of plasticizing agent/tanning agent subsequent to the wetting, but also facilitates that the proteins bindings sites are properly prepared when protein targeting agents are added.

It has thus been established during realistic test that a failure to perform an effective pre-wetting of the mycelium material may have an effect on the final strength properties of the processed mycelium material. If the mycelium material is finally processed/pressed into a fabric, e.g. applied in footwear, this lack of pre-wetting may materialize in that that fibres of the mycelium is not properly plasticized and consequently breaks during stress/use.

According to an embodiment of the invention, the wetting process step has a duration of 2 to 48 hours, such as 3 to 40 hours.

According to an embodiment of the invention, the wetting process is performed without performing mechanical compression on the mycelium material.

According to an embodiment of the invention, process liquid applied in the wetting process step includes water and a surfactant.

The surfactant is beneficial to the process as the wetting and the tanning process of the mycelium is intended to be as gentle as possible and thereby avoiding a tumbling process which is typically applied in a tanning process. A tumbling process represents a type of mechanical stress which in uncontrolled and the process may very likely damage the mycelium fibres. It is thus preferred that any mechanical process applied for assisting the present way of transporting process liquid into/through the mycelium fibres is carefully and predictably controlled so as to ensure that optional damage to the mycelium fibres are avoided or kept at a low level.

Surfactants are compounds that lower the surface tension (or interfacial tension) between two liquids, between a gas and a liquid, or between a liquid and a solid. In the present context, the surface tension between a liquid, typically water during the wetting process, is a particular concern in order to speed up the wetting process with little as possible use of mechanical stress to the mycelium.

Surfactants are usually organic compounds that are amphiphilic, meaning they contain both hydrophobic groups (their tails) and hydrophilic groups (their heads). Therefore, a surfactant typically contains both a water-insoluble (or oil-soluble) component and a water-soluble component. Surfactants will diffuse in water and adsorb at interfaces between the water (e.g. water applied during the wetting process) and the mycelium material.

Applicable surfactants include e.g. Invaderm LU, soap or other surfactant suitable for lowering the surface tension between the water/process liquid and the mycelium.

In an embodiment, the surfactant is only added during the wetting process in a separate process step of the tanning process and prior to the tanning process.

The wetting process may by performed by process liquid of water with 1 to 20% by weight of surfactant, such as 2 to 15% by weight of surfactant.'

According to an embodiment of the invention, the wetting process is followed by a subsequent tanning process step.

FIG. 17 illustrate a system of process tanks applicable for execution of the process of FIG. 16, namely a wetting tank WT, a plasticizing tank (and/or a tanning tank) PT/TT, and a dying tank DT. During these processes the thickness of the mycelium panel may remain constant or reduced somewhat, but still the same magnitude of thickness when resting.

The process step in the present embodiment is performed in different process tanks and the mycelium is moved from one process tank when a process step is finalized into another process tank where the next process step is to be performed. The movement between process tanks are indicated by movement steps 1, 2 and 3 (from a process tank to a compression arrangement CA.

All the illustrated process tanks may be configured with flow inducers according to any of the previously described and explained embodiments, mechanically induced compression and de-compression cycles and/or combinations of mechanically induced compression and decompression cycles may be combined with active establishment of liquid flow by flow inducers in the exterior process liquid.

Subsequent to dyeing, the mycelium panel is reduced by means of a compressing member CM reducing the thickness of the mycelium panel MP significantly into a mycelium sheet. The thickness may e.g. be reduced from 10 cm to 3-5 mm or even less.

A benefit of the above process tank system is that each tank may be optimized and controlled for a specific dedicated process step, e.g. the illustrated process step.

The above explained embodiment(s) in FIGS. 16 and/or 17 may comprise flow inducers and/or compression member according to of the FIGS. 2a to 7 and/or according to any of the FIGS. 8 to 15.

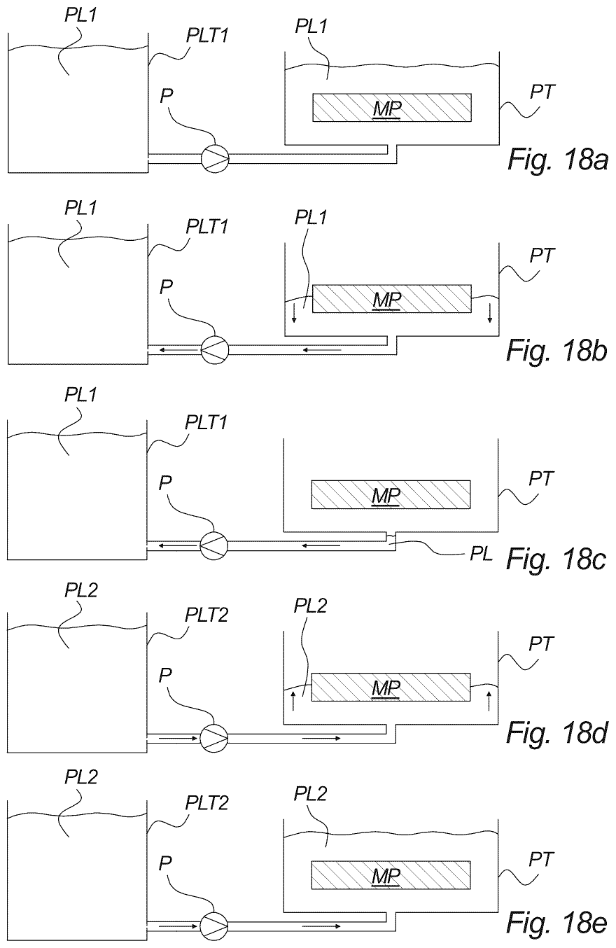

FIG. 18a-e illustrate the principle of a tank operation which may perform two or more of the mycelium processing steps of FIG. 16 in one process tank.

The illustrated process execution is a variant of the process flow applied in the process tank arrangement of FIG. 5a-5e, where the same process tank e.g. may be applied for a wetting process step in FIG. 18a by means of a first process liquid PL1. When the wetting process step is finished, first process liquid may be pumped out of the process tank PT into a first process liquid tank PLT1. When the process tank PT is empty, a second process liquid PL2, e.g. process liquid comprising plasticizing agent may be pumped into the process tank PT from a second process liquid tank PLT 2.

The process tank may comprise relevant valves and flow controllers to obtain and coupled the right tanks with each other and the process tank may further be fitted with combined flow assisting measures e.g. according to the embodiment of FIG. 15, flow assisting measures according to FIG. 6 or FIG. 13a.

Different further actions may be performed between the illustrated wetting process step WP and the plasticizing step PP. Such further actions may include rinsing, heating, steaming

### Actuators

An actuator in the present context is a component of a machine that is responsible for moving and controlling a mechanism or system, for example by opening a valve. In simple terms, it is a “mover”.

An actuator requires a control signal and a source of energy. The control signal is relatively low energy and may be electric voltage or current, pneumatic, or hydraulic fluid pressure, or even human power. Its main energy source may be an electric current, hydraulic pressure, or pneumatic pressure. When it receives a control signal, an actuator responds by converting the source's energy into mechanical motion. In the electric, hydraulic, and pneumatic sense, it is a form of automation or automatic control.

An actuator is a mechanism by which a control system acts upon to perform an operation or task. The control system can be simple (a fixed mechanical or electronic system) or software-based. It may typically be beneficial to apply a cooperating user interface by means of which a human operator may at least initiate a wetting process, stop the compression process in case of malfunctioning or react on different kinds of alarms and monitoring signals.

According to an advantageous embodiment of the invention at least one of the compression/de-compression cycles are performed automatically.

The pressure applied onto the mycelium material should preferably be monitored during at least the compression during the wetting process and the compression may be based on the measured compression force, so as to avoid destructive compression.

Destructive compression is in particular critical during the wetting process when compared to the compression later on during e.g. plastification, dying as the mycelium fibers are more brittle when non-saturated with water/moisture.

### Pumps

Pumps can be classified by their method of displacement into positive-displacement pumps, impulse pumps, velocity pumps, gravity pumps, steam pumps and valveless pumps. There are three basic types of pumps: positive-displacement, centrifugal and axial-flow pumps. In centrifugal pumps the direction of flow of the fluid changes by ninety degrees as it flows over impeller, while in axial flow pumps the direction of flow is unchanged.

Positive-displacement pumps might be applicable within the scope of the invention

A positive-displacement pump makes a fluid move by trapping a fixed amount and forcing (displacing) that trapped volume into the discharge pipe.

Some positive-displacement pumps may use an expanding cavity on the suction side and a decreasing cavity on the discharge side. Liquid flows into the pump as the cavity on the suction side expands and the liquid flows out of the discharge as the cavity collapses. The volume is constant through each cycle of operation.

Positive-displacement pumps, unlike centrifugal, can theoretically produce the same flow at a given speed (RPM: round per minute) no matter what the discharge pressure is. Thus, positive-displacement pumps are constant flow machines. However, a slight increase in internal leakage as the pressure increases prevents a truly constant flow rate.

A positive-displacement pump should preferably not operate against a closed valve on the discharge side of the pump, because it has no shutoff head like centrifugal pumps. It should therefore be avoided that the pump operates against a closed discharge valve.

A relief or safety valve on the discharge side of the positive-displacement pump may therefore be advantageous. The relief valve can be internal or external. The pump manufacturer normally has the option to supply internal relief or safety valves. The internal valve is usually used only as a safety precaution. An external relief valve in the discharge line, with a return line back to the suction line or supply tank provides increased safety of human and equipment both.

A positive-displacement pump can be further classified according to the mechanism used to move the fluid:
Rotary-type positive displacement: internal or external gear pump, screw pump, lobe pump, shuttle block, flexible vane or sliding vane, circumferential piston, flexible impeller, helical twisted roots (e.g. the Wendelkolben pump) or liquid-ring pumps
Reciprocating-type positive displacement: piston pumps, plunger pumps or diaphragm pumps
Linear-type positive displacement: rope pumps and chain pumps

A further type of pumps is rotary positive-displacement pumps applicable within the scope of the invention.

These pumps move fluid using a rotating mechanism that creates a vacuum that captures and draws in the liquid.
Advantages: Rotary pumps are very efficient[6] because they can handle highly viscous fluids with higher flow rates as viscosity increases.
Drawbacks: The nature of the pump requires very close clearances between the rotating pump and the outer edge, making it rotate at a slow, steady speed. If rotary pumps are operated at high speeds, the fluids cause erosion, which eventually causes enlarged clearances that liquid can pass through, which reduces efficiency.

Rotary positive-displacement pumps may include:
Gear pumps—a simple type of rotary pump where the liquid is pushed around a pair of gears.
Screw pumps—the shape of the internals of this pump is usually two screws turning against each other to pump the liquid
Rotary vane pumps
Hollow disk pumps (also known as eccentric disc pumps or Hollow rotary disc pumps), similar to scroll compressors, these have a cylindrical rotor encased in a circular housing. As the rotor orbits and rotates to some degree, it traps fluid between the rotor and the casing, drawing the fluid through the pump. It is used for highly viscous fluids like petroleum-derived products, and it can also support high pressures of up to 290 psi.
Vibratory pumps or vibration pumps are similar to linear compressors, having the same operating principle. They work by using a spring-loaded piston with an electromagnet connected to AC current through a diode. The spring-loaded piston is the only moving part, and it is placed in the center of the electromagnet. During the positive cycle of the AC current, the diode allows energy to pass through the electromagnet, generating a magnetic field that moves the piston backwards, compressing the spring, and generating suction. During the negative cycle of the AC current, the diode blocks current flow to the electromagnet, letting the spring uncompress, moving the piston forward, and pumping the fluid and generating pressure, like a reciprocating pump. Due to its low cost, it is widely used in inexpensive espresso machines. However, vibratory pumps cannot be operated for more than one minute, as they generate large amounts of heat. Linear compressors do not have this problem, as they can be cooled by the working fluid (which is often a refrigerant).

A further type of pumps is reciprocating positive-displacement pumps applicable within the scope of the invention.

Reciprocating pumps move the fluid using one or more oscillating pistons, plungers, or membranes (diaphragms), while valves restrict fluid motion to the desired direction. In order for suction to take place, the pump must first pull the plunger in an outward motion to decrease pressure in the chamber. Once the plunger pushes back, it will increase the chamber pressure and the inward pressure of the plunger will then open the discharge valve and release the fluid into the delivery pipe at constant flow rate and increased pressure.

Pumps in this category range from simplex, with one cylinder, to in some cases quad (four) cylinders, or more. Many reciprocating-type pumps are duplex (two) or triplex (three) cylinder. They can be either single-acting with suction during one direction of piston motion and discharge on the other, or double-acting with suction and discharge in both directions. The pumps can be powered manually, by air or steam, or by a belt driven by an engine. This type of pump was used extensively in the 19th century—in the early days of steam propulsion—as boiler feed water pumps. Now reciprocating pumps typically pump highly viscous fluids like concrete and heavy oils, and serve in special applications that demand low flow rates against high resistance. Reciprocating hand pumps were widely used to pump water from wells. Common bicycle pumps and foot pumps for inflation use reciprocating action.

These positive-displacement pumps have an expanding cavity on the suction side and a decreasing cavity on the discharge side. Liquid flows into the pumps as the cavity on the suction side expands and the liquid flows out of the discharge as the cavity collapses. The volume is constant given each cycle of operation and the pump's volumetric efficiency can be achieved through routine maintenance and inspection of its valves.

Typical reciprocating pumps include:
Plunger pumps—a reciprocating plunger pushes the fluid through one or two open valves, closed by suction on the way back.
Diaphragm pumps—similar to plunger pumps, where the plunger pressurizes hydraulic oil which is used to flex a diaphragm in the pumping cylinder. Diaphragm valves are used to pump hazardous and toxic fluids.
Piston pumps displacement pumps—usually simple devices for pumping small amounts of liquid or gel manually. The common hand soap dispenser is such a pump.
Radial piston pumps—a form of hydraulic pump where pistons extend in a radial direction.

Various positive-displacement pumps include:
Rotary lobe pump
Progressive cavity pump
Rotary gear pump
Piston pump
Diaphragm pump
Screw pump
Gear pump
Hydraulic pump
Rotary vane pump
Peristaltic pump
Rope pump
Flexible impeller pump

A further type of pumps is gear pumps applicable within the scope of the invention.

A gear pumps is the simplest form of rotary positive-displacement pumps. It may comprise of two meshed gears that rotate in a closely fitted casing. The tooth spaces trap fluid and force it around the outer periphery. The fluid does not travel back on the meshed part, because the teeth mesh closely in the center. Gear pumps see wide use in car engine oil pumps and in various hydraulic power packs.

A further type of pumps is screw pumps applicable within the scope of the invention.

A screw pump is a more complicated type of rotary pump that uses two or three screws with opposing thread—e.g., one screw turns clockwise and the other counterclockwise. The screws are mounted on parallel shafts that have gears that mesh so the shafts turn together and everything stays in place. The screws turn on the shafts and drive fluid through the pump. As with other forms of rotary pumps, the clearance between moving parts and the pump's casing is minimal.

A further type of pump applicable within the scope of the invention is a progressing cavity pump

This type of pump is widely used for pumping difficult materials, such as sewage sludge contaminated with large particles, this pump may comprise of a helical rotor, about ten times as long as its width. This can be visualized as a central core of diameter x with, typically, a curved spiral wound around of thickness half x, though in reality it is manufactured in a single casting. This shaft fits inside a heavy-duty rubber sleeve, of wall thickness also typically x. As the shaft rotates, the rotor gradually forces fluid up the rubber sleeve. Such pumps can develop very high pressure at low volumes.

A further type of pump applicable within the scope of protection is a roots lobe pump or root type pump.

As lobe pump is widely used for displacing the liquid trapped between two long helical rotors, each fitted into the other when perpendicular at 90°, rotating inside a triangular shaped sealing line configuration, both at the point of suction and at the point of discharge. This design produces a continuous flow with equal volume and no vortex. It may work at low pulsation rates, and offers gentle performance that may be required by some applications.

Typical applications include:
High capacity industrial air compressors.
Roots superchargers on internal combustion engines.
A brand of civil defense siren, the Federal Signal Corporation's Thunderbolt.

A further type of pumps is peristaltic pumps applicable within the scope of the invention.

A peristaltic pump is a type of positive-displacement pump. It contains fluid within a flexible tube fitted inside a circular pump casing (though linear peristaltic pumps have been made). A number of rollers, shoes, or wipers attached to a rotor compresses the flexible tube. As the rotor turns, the part of the tube under compression closes (or occludes), forcing the fluid through the tube. Additionally, when the tube opens to its natural state after the passing of the cam it draws (restitution) fluid into the pump. This process is called peristalsis and is used in many biological systems such as the gastrointestinal tract.

A further type of pumps is plunger pumps applicable within the scope of the invention.

Plunger pumps are reciprocating positive-displacement pumps.

These may include a cylinder with a reciprocating plunger. The suction and discharge valves are mounted in the head of the cylinder. In the suction stroke, the plunger retracts and the suction valves open causing suction of fluid into the cylinder. In the forward stroke, the plunger pushes the liquid out of the discharge valve. Efficiency and common problems could be: With only one cylinder in plunger pumps, the fluid flow varies between maximum flow when the plunger moves through the middle positions, and zero flow when the plunger is at the end positions. A lot of energy is wasted when the fluid is accelerated in the piping system. Vibration and water hammer may be a serious problem. In general, the problems may be compensated for by using two or more cylinders not working in phase with each other.

A further type of pumps is triplex-style plunger pumps applicable within the scope of the invention.

Triplex plunger pumps use three plungers, which reduces the pulsation of single reciprocating plunger pumps. Adding a pulsation dampener on the pump outlet can further smooth the pump ripple, or ripple graph of a pump transducer. The dynamic relationship of the high-pressure fluid and plunger generally requires high-quality plunger seals. Plunger pumps with a larger number of plungers have the benefit of increased flow, or smoother flow without a pulsation damper. The increase in moving parts and crankshaft load may be one drawback.

Durable high-pressure seals, low-pressure seals and oil seals, hardened crankshafts, hardened connecting rods, thick ceramic plungers and heavier duty ball and roller bearings improve reliability in triplex pumps. Triplex pumps now are in a myriad of markets across the world.

A further type of pumps is compressed-air-powered double-diaphragm pumps applicable within the scope of the invention.

A modem application of positive-displacement pumps is compressed-air-powered double-diaphragm pumps. Run on compressed air, these pumps are intrinsically safe by design, although all manufacturers offer ATEX certified models to comply with industry regulation. These pumps are relatively inexpensive and can perform a wide variety of duties, from pumping water out of bunds to pumping hydrochloric acid from secure storage (dependent on how the pump is manufactured—elastomers/body construction). These double-diaphragm pumps can handle viscous fluids and abrasive materials with a gentle pumping process ideal for transporting shear-sensitive media.[21]

A further type of pumps is rope pumps applicable within the scope of the invention.

A further type of pumps is Impulse pumps applicable within the scope of the invention.

Impulse pumps use pressure created by gas (usually air). In some impulse pumps the gas trapped in the liquid (usually water), is released and accumulated somewhere in the pump, creating a pressure that can push part of the liquid upwards.

Conventional impulse pumps include:
Hydraulic ram pumps—kinetic energy of a low-head water supply is stored temporarily in an air-bubble hydraulic accumulator, then used to drive water to a higher head.
Pulser pumps—run with natural resources, by kinetic energy only.
Airlift pumps—run on air inserted into pipe, which pushes the water up when bubbles move upward

Instead of a gas accumulation and releasing cycle, the pressure can be created by burning of hydrocarbons. Such combustion driven pumps directly transmit the impulse from a combustion event through the actuation membrane to the pump fluid. In order to allow this direct transmission, the pump needs to be almost entirely made of an elastomer (e.g. silicone rubber). Hence, the combustion causes the membrane to expand and thereby pumps the fluid out of the adjacent pumping chamber.

A further type of pumps is hydraulic ram pumps applicable within the scope of the invention.

A hydraulic ram is a water pump powered by hydropower. It takes in water at relatively low pressure and high flow-rate and outputs water at a higher hydraulic-head and lower flow-rate. The device uses the water hammer effect to develop pressure that lifts a portion of the input water that powers the pump to a point higher than where the water started.

The hydraulic ram is sometimes used in remote areas, where there is both a source of low-head hydropower, and a need for pumping water to a destination higher in elevation than the source. In this situation, the ram is often useful, since it requires no outside source of power other than the kinetic energy of flowing water.

A further type of pumps is velocity pumps applicable within the scope of the invention.

Rotodynamic pumps (or dynamic pumps) are a type of velocity pump in which kinetic energy is added to the fluid by increasing the flow velocity. This increase in energy is converted to a gain in potential energy (pressure) when the velocity is reduced prior to or as the flow exits the pump into the discharge pipe. This conversion of kinetic energy to pressure is explained by the First law of thermodynamics, or more specifically by Bernoulli's principle.

Dynamic pumps may further be subdivided according to the means in which the velocity gain is achieved.

These types of pumps may include a number of characteristics:
Continuous energy
Conversion of added energy to increase in kinetic energy (increase in velocity)
Conversion of increased velocity (kinetic energy) to an increase in pressure head

A practical difference between dynamic and positive-displacement pumps is how they operate under closed valve conditions. Positive-displacement pumps physically displace fluid, so closing a valve downstream of a positive-displacement pump produces a continual pressure build up that can cause mechanical failure of pipeline or pump. Dynamic pumps differ in that they can be safely operated under closed valve conditions (for short periods of time).

A further type of pumps is radial-flow pumps applicable within the scope of the invention.

Such a pump is also referred to as a centrifugal pump. The fluid enters along the axis or center, is accelerated by the impeller and exits at right angles to the shaft (radially); an example is the centrifugal fan, which is commonly used to implement a vacuum cleaner. Another type of radial-flow pump is a vortex pump. The liquid in them moves in tangential direction around the working wheel. The conversion from the mechanical energy of motor into the potential energy of flow comes by means of multiple whirls, which are excited by the impeller in the working channel of the pump. Generally, a radial-flow pump operates at higher pressures and lower flow rates than an axial- or a mixed-flow pump.

A further type of pumps is Axial-flow pumps applicable within the scope of the invention.

These are also referred to as All fluid pumps. The fluid is pushed outward or inward to move fluid axially. They operate at much lower pressures and higher flow rates than radial-flow (centrifugal) pumps. Axial-flow pumps cannot be run up to speed without special precaution. If at a low flow rate, the total head rise and high torque associated with this pipe would mean that the starting torque would have to become a function of acceleration for the whole mass of liquid in the pipe system. If there is a large amount of fluid in the system, accelerate the pump slowly.

Mixed-flow pumps function as a compromise between radial and axial-flow pumps. The fluid experiences both radial acceleration and lift and exits the impeller somewhere between 0 and 90 degrees from the axial direction. As a consequence mixed-flow pumps operate at higher pressures than axial-flow pumps while the might deliver higher discharges than radial-flow pumps. The exit angle of the flow dictates the pressure head-discharge characteristic in relation to radial and mixed-flow.

A further type of pumps is regenerative turbine pumps applicable within the scope of the invention.

Might also be known as drag, friction, liquid-ring, peripheral, side-channel, traction, turbulence, or vortex pumps, regenerative turbine pumps are class of rotodynamic pump that operates at high head pressures, typically 4-20 bars (4.1-20.4 kgf/cm2; 58-290 psi).

The pump include an impeller with a number of vanes or paddles which spins in a cavity. The suction port and pressure ports are located at the perimeter of the cavity and are isolated by a barrier called a stripper, which allows only the tip channel (fluid between the blades) to recirculate, and forces any fluid in the side channel (fluid in the cavity outside of the blades) through the pressure port. In a regenerative turbine pump, as fluid spirals repeatedly from a vane into the side channel and back to the next vane, kinetic energy is imparted to the periphery,[27] thus pressure builds with each spiral, in a manner similar to a regenerative blower.

As regenerative turbine pumps cannot become vapor locked, they are commonly applied to volatile, hot, or cryogenic fluid transport. However, as tolerances are typically tight, they are vulnerable to solids or particles causing jamming or rapid wear. Efficiency is typically low, and pressure and power consumption typically decrease with flow. Additionally, pumping direction can be reversed by reversing direction of spin.

A further type of pumps is eductor-jet pump applicable within the scope of the invention.

This uses a jet, often of steam, to create a low pressure. This low pressure sucks in fluid and propels it into a higher pressure region.

A further type of pumps is gravity pumps applicable within the scope of the invention.

Gravity pumps include the syphon and Heron's fountain. The hydraulic ram is also sometimes called a gravity pump; in a gravity pump the water is lifted by gravitational force and so called gravity pump

A further type of pumps is steam pumps applicable within the scope of the invention.

Steam pumps have been for a long time mainly of historical interest. They include any type of pump powered by a steam engine and also pistonless pumps such as Thomas Savery's or the Pulsometer steam pump.

Recently there has been a resurgence of interest in low power solar steam pumps for use in smallholder irrigation in developing countries. Previously small steam engines have not been viable because of escalating inefficiencies as vapour engines decrease in size. However the use of modern engineering materials coupled with alternative engine configurations has meant that these types of system are now a cost-effective opportunity.

A further type of pumps is valveless pumps applicable within the scope of the invention.

Valveless pumping assists in fluid transport in various biomedical and engineering systems. In a valveless pumping system, no valves (or physical occlusions) are present to regulate the flow direction. The fluid pumping efficiency of a valveless system, however, is not necessarily lower than that having valves. In fact, many fluid-dynamical systems in nature and engineering more or less rely upon valveless pumping to transport the working fluids therein. For instance, blood circulation in the cardiovascular system is maintained to some extent even when the heart's valves fail. Meanwhile, the embryonic vertebrate heart begins pumping blood long before the development of discernible chambers and valves. In microfluidics, valveless impedance pumps have been fabricated, and are expected to be particularly suitable for handling sensitive biofluids. Ink jet printers operating on the piezoelectric transducer principle also use valveless pumping. The pump chamber is emptied through the printing jet due to reduced flow impedance in that direction and refilled by capillary action.

1. A mycelium process tank system, the process tank system comprising:
at least one mycelium support base,
a mycelium storing volume in a process tank,
a liquid storing volume fluidly coupled to and/or being a part of the process tank,
the liquid storing volume designating a volume exterior to physical boundaries of the mycelium storing volume,
the mycelium process tank comprising one or more pumps controlled by a pump controller, the one or more pumps being arranged for circulation of process liquid outside the mycelium storing volume in the process tank.

2. (canceled)

3. The mycelium process tank system according to claim 1, wherein a part of the process liquid forming interior process liquid is located within physical boundaries of a mycelium panel positioned in the mycelium storing volume.

4. (canceled)

5. The mycelium process tank system according to claim 1, wherein the circulation of process liquid outside the mycelium storing volume is obtained by means of activation of one or more pumps located in the process tank and/or coupled to the process tank.

6-8. (canceled)

9. The mycelium process tank system according to claim 1, wherein the process tank comprises one or more process liquid inputs and one or more process liquid outputs and wherein the one or more liquid inputs and/or the one or more liquid outputs are coupled to a pump, the pump establishing a flow between said one or more liquid inputs and one or more liquid outputs.

10-16. (canceled)

17. An industrial process of processing a mycelium panel, the process comprising:
providing a mycelium panel,
positioning the mycelium panel in a process tank,
inputting process liquid into the process tank, a part of the process liquid in the process tank forming exterior process liquid being located outside the physical boundaries of the mycelium panel, and
actively establishing a liquid flow in the exterior process liquid, wherein the liquid flow is established in proximity to at least a part of the physical boundaries of the mycelium panel.

18. The industrial process according to claim 17, wherein the mycelium panel is subsequently compressed into a non-woven fabric.

19. The industrial process according to claim 17, wherein the mycelium panel is subjected to a wetting process step prior to a plasticizing process step and/or a tanning process step.

20. The industrial process according to claim 19, wherein the wetting process step includes subjecting the mycelium panel to process liquid in the form of water prior to the tanning process step.

21. The industrial process according to claim 19, wherein the mycelium panel is submerged in the process liquid at least a part of the duration of the wetting process step.

22. (canceled)

23. The industrial process according to claim 19, wherein the wetting process step has a duration of 1 to 72 hours.

24. (canceled)

25. The industrial process according to claim 19, wherein the wetting process step is performed without performing mechanical compression on the mycelium panel.

26. (canceled)

27. The industrial process according to claim 19, wherein the wetting process step is followed by a subsequent tanning process step.

28. The industrial process according to claim 19, wherein the mycelium panel is submerged in the process liquid at least a part of the duration of the tanning process step.

29. The industrial process according to claim 19, wherein the mycelium panel comprises between 50 and 90% water by weight prior to the wetting process step.

30-32. (canceled)

33. The industrial process according to claim 17, wherein the mycelium panel is a submerged in the process liquid in at least one process tank during the process, and wherein, in an initial part of the process, the process tank being a wetting tank, and in a subsequent part of the process, the process tank being a plasticizing tank, a tanning tank, and/or a dyeing tank.

34. The industrial process according to claim 19, wherein the mycelium panel is oriented in the horizontal direction during the wetting process step, the plasticizing process step, the tanning process step, and/or the dyeing process.

35-37. (canceled)

38. The industrial process according to claim 19, wherein the processed mycelium panel is compressed into a final non-woven fabric subsequent to the wetting process step and the plasticizing process step.

39. The industrial process according to claim 19, wherein the processed mycelium panel is compressed into a final non-woven fabric subsequent to the wetting process step, the plasticizing process step and the dyeing process step.

40-42. (canceled)

43. The industrial process according to claim 17, wherein the inputting of process liquid is performed prior to, subsequent to and/or at the same time as the mycelium panel is positioned in the process tank.

44. The industrial process according to claim 17, wherein the filling and/or a draining of the process tank is performed by means of at least one pump.

45. (canceled)
